(19) United States
(12) Patent Application Publication
(10) Pub. No.: US 2025/0144429 A1
(43) Pub. Date: May 8, 2025

(54) OPTIMIZATION OF DBS PROGRAM USING A PRESPECIFIED SELECTION OF CONTACTS

(71) Applicant: Boston Scientific Neuromodulation Corporation, Valencia, CA (US)

(72) Inventors: Mahsa Malekmohammadi, Sherman Oaks, CA (US); Richard Mustakos, Simi Valley, CA (US); G. Karl Steinke, Valencia, CA (US)

(73) Assignee: Boston Scientific Neuromodulation Corporation, Valencia, CA (US)

(21) Appl. No.: 18/934,118

(22) Filed: Oct. 31, 2024

Related U.S. Application Data
(60) Provisional application No. 63/595,572, filed on Nov. 2, 2023.

(51) Int. Cl. A61N 1/36

(52) U.S. Cl. CPC A61N 1/36185; A61N 1/36157

(57) ABSTRACT

Methods and systems for planning configurations of a neuromodulation system having a lead with a plurality of electrodes. Configurations are planned using an optimizer that analyzes potential fractionalizations after removing unavailable electrodes from analysis and adjusting a candidate fractionalization to account for the removed electrodes. The unavailable electrodes may be identified by a physician or by analysis of impedances of the electrodes.

### CROSS-REFERENCE TO RELATED APPLICATIONS

The present application claims the benefit of U.S. Provisional Patent Application Ser. No. 63/595,572, filed Nov. 2, 2023, the disclosure of which is incorporated herein by reference.

### BACKGROUND

Deep brain stimulation (DBS) is a form of neuromodulation in which electrodes are implanted into the brain of a patient and used to deliver stimuli. Therapies directed to a variety of ailments, including Alzheimer's Disease, Parkinson's Disease, cognitive and/or memory decline, depression and other disorders have been proposed and/or implemented. Each patient has unique anatomy, and each treated ailment may call for different portions of the brain to receive therapy. As a result, accurate and precise targeting of therapy is desired.

For various reasons, one or more electrodes in a given implanted system may become unavailable for therapy delivery. For example, impedance at each electrode is typically monitored during use and compared to an impedance range; if the impedance at a given electrode goes out of range, that electrode may be disabled and marked as unusable by the system. High impedance, for example, may indicate fracture of a conductor that provides electrical connection between a pulse generator and the electrode. A physician may decide to mark an electrode as unusable if desired. A typical response to an electrode becoming unavailable may include, for example, disabling any therapy program that uses the electrode, as an anode or cathode. New and alternative methods and system for more rigorously handling an unusable electrode in therapy planning are desired.

### Overview

The present inventors have recognized, among other things, that a problem to be solved is the need for new and/or alternative methods and system for more rigorously handling an unusable electrode in therapy planning are desired. In an illustrative example, therapy planning is modified to adjust steering and current allocation strategies during optimization of therapy target setting processes.

A first illustrative and non-limiting example takes the form of a configuration system for configuring delivery of neuromodulation to specific tissue of a patient by an implantable system having a plurality of electrodes, the configuration system comprising: a receiver module configured to receive at least patient data for a patient and lead position indicating a location of a lead carrying at least two of the plurality of electrodes thereon relative to neural tissue of the patient; an optimizer for identifying candidate therapy parameter sets indicating utilization of the plurality of electrodes during therapy, the optimizer comprising: a steering module for identifying a potential fractionalization to analyze, the potential fractionalization setting proportions of total current to be issued by each of the electrodes; an electrode removal module that receives the potential fractionalization and removes unavailable electrodes from the potential fractionalization to generate a reduced fractionalization; an adjusting module that receives the reduced fractionalization and creates an adjusted fractionalization; a scoring module that receives the adjusted fractionalization and calculates a plurality of metrics for the adjusted fractionalization using a plurality of amplitude settings; and a candidate module that identifies one or more candidate therapies using the plurality of metrics.

Additionally or alternatively, the optimizer is configured to use the steering module to identify a plurality of potential fractionalizations.

Additionally or alternatively, the potential fractionalization identifies active and inactive electrodes, and the adjusting module creates the adjusted fractionalization by proportionally increasing a fraction of current to be delivered by each active electrode which is not removed by the electrode removal module.

Additionally or alternatively, the potential fractionalization identifies active and inactive electrodes, and the adjusting module modifies current fractionalization by adding current to active electrodes in proportion to proximity to the unavailable electrodes. Additionally or alternatively, the proportion is based on a Gaussian curve.

Additionally or alternatively, the electrode removal module removes electrodes having an impedance outside of an impedance range.

Additionally or alternatively, the electrode removal module removes electrodes that have been identified as unavailable by a physician.

Additionally or alternatively, the electrode removal module is configured to identify an electrode that could be removed by identifying proximity to a neural structure less than a threshold proximity, and suggesting electrode removal to a physician via a user interface.

Additionally or alternatively, the electrode removal module is configured to identify an electrode that could be removed by identifying an electrode having an impedance outside of an impedance range, and suggesting electrode removal to a physician via a user interface.

Additionally or alternatively, the system may also include a voxel calculation module which identifies volumes located around the lead as voxels, and labels at least some voxels as target voxels to which therapy is to be directed.

Additionally or alternatively, the voxel calculation module also labels at least some voxels as avoid voxels to which therapy is to be avoided.

Additionally or alternatively, wherein the scoring module calculates the plurality of metrics by determining which of the voxels would be activated by the adjusted fractionalization at a plurality of current amplitudes.

Additionally or alternatively, the scoring module uses one or more of a weight for target voxels, a weight for avoid voxels, a weight for total activated voxels and/or a weight for total activated non-target and non-avoid voxels to calculate the metrics.

Additionally or alternatively, the lead is configured to be implanted in the brain of the patient. Additionally or alternatively, the patient data includes anatomy data indicating actual or likely brain structure locations.

Another illustrative and non-limiting example takes the form of a method of operation in a neuromodulation system, the neuromodulation system including an implantable system having a plurality of electrodes, and a configuration system configured to perform steps of the method, the method comprising: a) receiving at least patient data for a patient and a lead position indicating a location of a lead carrying at least two of the plurality of electrodes thereon relative to neural tissue of the patient; b) identifying a potential fractionalization to analyze, the potential fractionalization setting proportions of total current to be issued by each of the electrodes; c) removing unavailable electrodes from the potential fractionalization to generate a reduced fractionalization; d) adjusting the reduced fractionalization to create an adjusted fractionalization; e) calculating a plurality of metrics for the adjusted fractionalization using a plurality of amplitude settings; and identifying one or more of the adjusted fractionalizations as one or more candidate therapies using the plurality of metrics.

Additionally or alternatively, the method also includes communicating the one or more candidate therapies from the configuration system to a pulse generator for the implantable system, and activating the pulse generator to issue neuromodulation to the patient using at least one of the one or more candidate therapies.

Additionally or alternatively, the method also includes identifying a plurality of potential fractionalizations, and performing each of c), d) and e) for each of the plurality of potential fractionalizations.

Additionally or alternatively, the potential fractionalization identifies active and inactive electrodes, and step d) includes proportionally increasing a fraction of current to be delivered by each active electrode which is has not removed during step c).

Additionally or alternatively, the potential fractionalization identifies active and inactive electrodes, and step d) include increasing proportions of total current to be issued by each of the electrodes in proportion to proximity to the unavailable electrodes. Additionally or alternatively, the proportion is based on a Gaussian curve.

Additionally or alternatively, step c) includes removing electrodes having an impedance outside of an impedance range.

Additionally or alternatively, step c) includes removing electrodes that have been identified as unavailable by a physician.

Additionally or alternatively, step c) includes identifying an electrode as potentially unavailable based on proximity to a neural structure less than a threshold proximity, and presenting, to a physician via a user interface, a suggestion to remove the potentially unavailable electrode.

Additionally or alternatively, step c) is performed by identifying an electrode that could be removed by identifying an electrode having an impedance outside of an impedance range, and suggesting electrode removal to a physician via a user interface.

Additionally or alternatively, step e) includes: identifying volumes located around the lead as voxels; labeling at least some voxels as target voxels to which therapy is to be directed; and labeling at least some voxels as avoid voxels to which therapy is to be avoided.

Additionally or alternatively, step e) includes determining which of the voxels would be activated by the adjusted fractionalization at a plurality of current amplitudes.

Additionally or alternatively, step e) includes applying one or more of a weight for target voxels, a weight for avoid voxels, and a weight for total activated voxels.

Additionally or alternatively, the lead is implanted in the brain of the patient, and the patient data includes anatomy data indicating actual or likely brain structure locations.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative DBS system implanted in a patient;

FIG. 2 illustrates details of a generic directional DBS lead;

FIG. 3 illustrates a display of lead electrodes;

FIG. 4 shows an illustrative method in block form;

FIG. 5 shows an illustrative optimization method in block form; and

FIG. 6 shows an illustrative system in block form.

### DETAILED DESCRIPTION

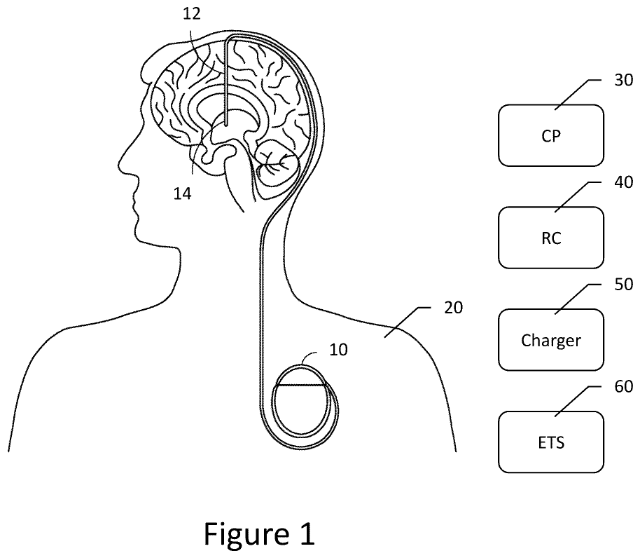

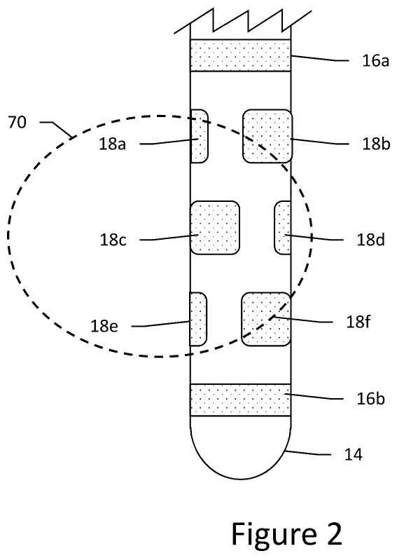

FIG. 1 shows an illustrative DBS system implanted in a patient. The system comprises a pulse generator 10, shown implanted in the upper torso of a patient 20. The pulse generator 10 is coupled to a lead 12, which extends subcutaneously to the head of the patient 20, through a burr hole formed in the patient's skull, and then into the brain of the patient. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulata, the cortex, the globus pallidus externus, the medial forebrain bundle, the periaqueductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, and avoidance regions or “Avoid” regions are brain structures associated with side effects.

Conditions to be treated may include dementia, Alzheimer's disease, Parkinson's disease, various tremors, depression, anxiety or other mood disorders, sleep related conditions, etc. Therapeutic benefits may include, for example, and without limitation, improved cognition, alertness, and/or memory, enhanced mood or sleep, avoidance of pain or tremor, reduction in motor impairments, and/or preservation of existing function and/or cellular structures, such as preventing loss of tissue and/or cell death. Side effects can include a wide range of issues such as, for example, and without limitation, reduced cognition, alertness, and/or memory, degraded sleep, depression, anxiety, unexplained weight gain/loss, tinnitus, pain, tremor, etc. Therapeutic benefits and side effects may be monitored using, for example, patient surveys, performance tests, and/or physical monitoring such as monitoring gait, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic, monophasic with passive recovery, and other stimulation types may be used as desired. The CP 30 may be, for example, a laptop or tablet computer and can be used by a physician, or at the direction of a physician, to obtain data from and provide instructions to the pulse generator 10 via suitable communications protocols such as Bluetooth or MedRadio or other wireless communications protocols, and/or via other modalities such as inductive telemetry.

A patient remote control (RC) 40 can be used by the patient to perform various actions relative to the pulse generator 10. These may be physician defined options, and may include, for example, turning therapy on and/or off, making (limited) adjustments to therapy such as selecting from available therapy programs and adjusting amplitude settings, and/or entering requested information, such as answer questions about activities and therapy benefits and side effects. The RC 40 can communicate via similar telemetry as the CP 30 to control and/or obtain data from the pulse generator 10. The patient RC 40 may be programmable on its own, or may communicate or be linked with the CP 30.

A charger 50 may be provided to the patient to allow the patient to recharge the pulse generator 10, if the pulse generator 10 is rechargeable. Some pulse generators 10 are not rechargeable, and so the charger 50 may be omitted. The charger 50 can operate, for example, by generating a varying magnetic field to activate an inductor associated with the pulse generator 10 to provide power to recharge the pulse generator, using known methods.

Some systems may include an external test stimulator (ETS) 60. The ETS 60 can be used intraoperatively to test therapy programs after the lead 12 has been positioned in the patient to test therapy for the patient 20. For example, an initial implantation of the lead 12 can take place using, for example, a stereotactic guidance system, with the pulse generator 10 temporarily left out. The lead 12 may have a proximal end thereof connected to an intermediate connector (sometimes called an operating room cable) that couples to the ETS 60. After lead 12 has been implanted and coupled to the ETS 60, the ETS 60 can be programmed using the CP 30 with various therapy programs and stimulation parameters, which are tested to determine therapy effects. Lead position may be adjusted, as needed during this process. Once therapy suitability for the patient is established, the permanent pulse generator 10 is implanted and the lead 12 is connected thereto, with the ETS 60 then being removed from use.

The pulse generator 10 may include operational circuitry for generating output stimulation programs and/or pulses in accordance with stored instructions. Some examples of current or prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of the pulse generator 10, CP 30, RC 40, Charger 50, and ETS 60 can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

FIG. 2 illustrates details of a generic directional DBS lead. The distal end 14 carrying a plurality of electrodes are shown. Two ring electrodes 16a, 16b (collectively ring electrodes 16) can be provided as shown, and a number of segmented electrodes are shown at 18a, 18b, 18c, 18d, 18e, 18f (collectively, segmented electrodes 18). Each electrode 16, 18 is separately addressable in the system, such as by using a pulse generator having multiple independent current control (MICC), or multiple voltage sources,

MICC is a stimulus control system that provides a plurality of independently generated output currents that may each have an independent quantity of current. The use of MICC can allow spatially selective fields to be created by therapy outputs. The term “fractionalization” may refer to how the total current issued by the pulse generator via the electrodes is divided up amongst the electrodes 16, 18.

The pulse generator canister may be used, for example, as a return electrode during therapy outputs. If desired, one of the lead electrodes (such as a ring electrode 16 or one or more of the segmented electrodes 18) may instead be used as a return electrode. Thus, for example, the electrodes on the lead may serve as cathodes while pulse generator canister serves as an anode during one phase of stimulation pulse delivery (which may be reversed in another phase of stimulation pulse delivery). In another example, some of the lead electrodes 16, 18 serve as cathodes, while other lead electrodes serve as anodes during one phase of stimulation pulse delivery. Any suitable combination and quantity of anodes and cathodes may be used for therapy purposes, and any lead electrode and/or the housing electrode can be used in any of these roles, as needed.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

MICC used with a directional lead can facilitate precise therapy targeting. For example, a directional lead as shown in FIG. 2 may be used to generate a stimulation field as illustrated at 70 in FIG. 2. The outer boundary of field 70 may be understood as representing an equipotential or equal field boundary, within which the electrical field is higher than an activation threshold, and outside of which the electrical field is below the threshold, for purposes of illustration. An activation threshold may represent or approximate a voltage/field threshold at which neural cells will activate or “fire”. Activation thresholds may be determined on a population basis, such as by relating to a voltage/field at which a 50% likelihood of activation of 50% of the cell population is determined, thought other boundaries/thresholds can be used. The shape of the field can be adjusted, as described in the references incorporated by reference above, by modifying the fractionalization of current issued via the electrodes using MICC. An output creating an activation field boundary as shown at 70 may be (roughly) generated by using electrode 18c as a cathode, and surrounding electrodes 18a and, 18e as anodes, for example. Actual characteristics of fractionalization may be more sophisticated than this simple example.

The boundary shown at 70 in FIG. 2 can be used to illustrate stimulation field effects, and can be generated for purposes of display using stimulation field modeling (SFM). In SFM, the tissue is modeled, for example, using finite element models in which the lead body is treated as an insulator, surrounded by a thin encapsulation sheath, and surrounded by neural tissue. Neural tissue may be modeled as isotropic and homogenous, though more sophisticated modelling can also be used if desired. For SFM, a set of model voxels are defined around the lead, breaking up the volume into small segments, each of which can be analyzed within the model. The outer boundaries of the SFM can be determined using a population-based activation threshold as described above. The result can be that at a given fractionalization and total stimulation current, an SFM can be generated as a three-dimensional surface surrounding a portion of the lead and encompassing a volume of neural tissue. Field 70 may be, for example, understood as a two-dimensional representation of a slice of the SFM. As noted, the SFM can be used as a visual tool for illustrating, to a patient or physician, what tissue is or is not being stimulated by a given fractionalization and total current.

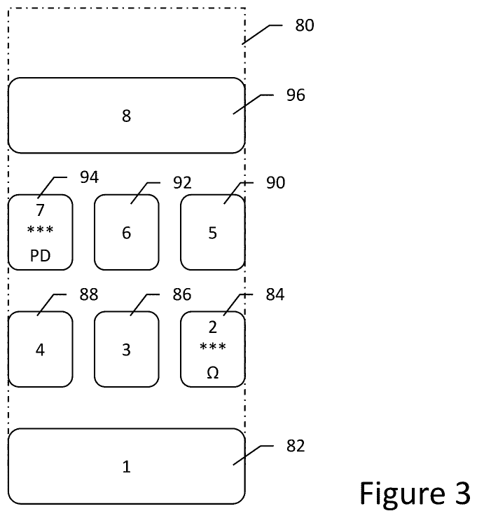

FIG. 3 shows another representation of a set of electrodes on a lead. The lead is illustrated in broken lines at 80. Each electrode on the lead is numbered as shown at 82, 84, 86, 88, 90, 92, 94, 96. Some of the electrodes are shown with a set of asterisks (***) indicating that those electrodes 84, 94 have been marked as unavailable, with the basis of this lack of availability also shown. This means that the pulse generator 10 (FIG. 1) and its programming via the CP 30 (FIG. 1), will not allow those electrodes 84, 94, to be used in any therapy program for receiving or issuing currents or voltages.

Electrode 84, for example, is marked unavailable with the asterisks (***), and the reason is an out of range impedance as indicated by the Ω. For example, each electrode may be tested to determine impedance that a current passing through the electrode experiences. That tested impedance is compared to an acceptable range. As the skilled person would understand, there may be many different reasons for an electrode impedance to be out of range. For example, if a current controlled system is used, an impedance that is too high may prevent therapy delivery as the constant current circuitry may require a voltage source that is larger than the electronics can support. High impedance may occur due to conductor fracture within the lead 80, or due to other causes such as weld failure at the location where the conductor is electrically connected to the electrode itself. High impedance may occur as well due to excess corrosion on the electrode-tissue interface. Unduly low impedance may occur due to shorting somewhere in the lead or fluid incursion into an undesired portion of the pulse generator header where the lead couples to the pulse generator, for example. Voltage field measurements may be made and used similarly, such as by issuing an output current or voltage between the ring electrodes 82, 96, and sensing electric field that results on the rest of the electrodes 84-94 to confirm connection of the electrodes to the pulse generator's internal circuitry.

Electrode 94 is also marked as unavailable, but this time due to physician decision, thus the marking “PD”. For example, the physician may determine, on review of images taken after lead implantation, that the electrode 94 is located adjacent or too near to a structure that the physician does not want subjected to electrical fields. The CP (FIG. 1, at 30) may include a user interface and screens allowing the physician to make such choices.

Prior systems have not offered rigorous approaches to handling unavailable electrodes. For example, if an electrode fails impedance testing, the matter is initially addressed via a physician alert. Any program using that electrode for therapy delivery may be automatically disabled by the pulse generator, for example. When the patient is next in-clinic and programming is performed by the physician, manual reprogramming may be required, for example. New and alternative methods are desired.

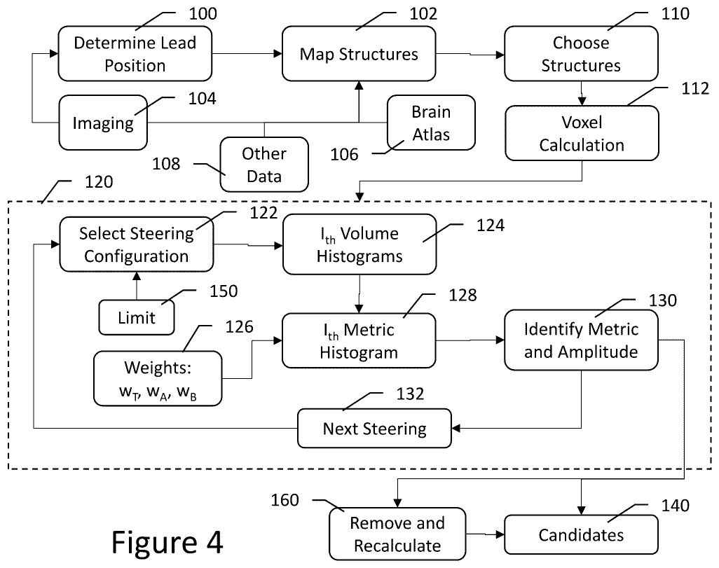

FIG. 4 shows an illustrative method in block form. In the illustrative method, lead position in the patient is determined at 100. For purposes of this illustration, lead position in the brain will be explained, however, the present invention may be used when modeling and optimizing neural stimulation in other parts of the body. The lead position 100 may be determined by use of imaging 104, such as using various modalities that may include X-ray, CT scan, MRI, or others, after the lead has been implanted.

Next, the system maps structures, as indicated at 102. Mapping structures 102 may include the use of pre-operative and/or post-operative imaging 104 and data from a brain atlas 106 to identify structures within the brain. A brain atlas 106 may include data from a population of patients indicating the general position and nature of structures in the brain, allowing the images to be referenced against population examples. Illustrative structures may include the thalamus, the globus pallidus, the subthalamic nucleus, and/or other structures listed above, and data identifying such structures actual (from imaging) or estimated (from population/atlas, for example) may be referred to as patient anatomy data. Data input to 102 may also include other data 108 such as, for example and without limitation, inputs from a database of therapy settings of a previously programmed/treated population of patients, as desired. That is, therapy issued by other implantable systems may target similar structures repeatedly and so may provide additional understanding of, for example, best practices. Other data 108 may also include brain functional data such as gathered using functional-based imaging, or electrophysiological activities recorded using an implanted lead, each of which may also enter the system as data input at 102. This collective data is used to map locations of structures in the brain, as well as “sweet” and “sour” spots-areas associated with therapeutic benefits and/or harms or side-effects.

The user or physician then chooses structures at block 110. Structures may be identified as target structures or avoid structures. Target structures are those that the physician determines should be stimulated as much as possible, and avoid structures are those that the physician determines should not be stimulated, to the extent possible. Typically, target structures are associated with therapy benefits, and avoid structures are those that are associated with side effects.

Voxel calculation occurs as indicated at 112. The voxel calculation 112 defines a grid of volume elements (voxels) in the tissue region around the lead, and determines which voxels are in various structures, as further described below. As used herein, “voxel” refers to any segment of volume used in an analysis, regardless of shape and may include cubes, polygons, partial cylinders, partial toroidal shapes, etc. Voxel calculation may reference any of world, anatomical or image coordinate systems. Any suitable voxel definition and coordinate system (such as Cartesian, spherical, or polar coordinates) can be used if desired. Some systems may use an anatomical reference for the relevant coordinate system to define both lead position and structure positions/locations. The skilled person will understand transformations from one coordinate system to another. The voxel calculation 112 includes identification of which target and avoid structures contains which voxels, using the choices made at block 110. A single voxel may be in multiple structures.

An optimization follows in an iterative block 120. The structure choices 110 and voxel calculation 112, and/or device history or other inputs, are used to determine an initial steering or fractionalization at steering configuration 122. The steering configuration 122 is then used to determine an Iₜₕ table. The contents of the Iₜₕ table indicate, for each voxel using a given steering state and fractionalization, the minimum total current that would be needed or likely to trigger neural activity in the given voxel. The values of the Iₜₕ table are used to create Iₜₕ Volume Histograms 124. For each target or avoid structure, an Iₜₕ Volume Histogram is created, and contains bins for each of the available amplitude settings (voltage or current levels). Each voxel is characterized as activated or not activated at a plurality of amplitudes, using the Iₜₕ table data. Relative to each target or avoid structure, the voxel has a “value” that is calculated as further discussed below, where the voxel value indicates, in part, the fraction of the voxel that is inside a particular structure. Each bin has a value and associated amplitude, where the bin value is determined by summing the product of the voxel volume, times voxel value, for each voxel that would be activated at the change from the amplitude below that of the bin, to the amplitude for the bin. That is, each bin in an Iₜₕ Volume Histogram specifies the change in stimulated volume of each structure at each of a range of amplitudes for the steering and other therapy parameters used to generate the Iₜₕ table.

The contents of these Iₜₕ Volume Histograms 124, and the target/avoid region selections, are combined with weights 126, to generate the Iₜₕ Metric Histogram 128. In a simple approach, there may be two user-adjustable weights, and one pre-set weight: target volume weight, w_{T} may be pre-set to 1, and each of an avoid structure weight, w_{A}, and background weight w_{B} may be user adjustable; other approaches to weighting for target, avoid and background structures may be used. The Iₜₕ Metric Histogram 128 thus indicates, for each of a plurality of amplitudes, the resultant per-amplitude change in metric based on which voxels that are activated and within a target or avoid region, as weighted in accordance with weights 126.

The product of the weighting values 126 of each voxel and the Iₜₕ Volume Histograms 124 is referred to as an Iₜₕ Metric Histogram 128. The per-amplitude change in metric values are integrated to determine the highest metric value and the associated amplitude that generates the highest metric value for the steering configuration under analysis, at block 130. These values, and those generated by previous iterations of the optimization, are used to generate a next steering state, prompting the next iteration, as indicated at 132. If an exit condition is met, such as by showing that the metric is not increasing with new steering configurations, the iterations in 120 terminate and the resulting candidates, including the various maximum metrics and amplitudes, are analyzed at 140.

During the analysis in FIG. 4, each voxel may be understood as having its own value, depending on whether the voxel is in a target region or avoid region. The background, as used below, is the entire volume that the model predicts would be stimulated, including target and avoid regions as well as voxels that are in neither target nor avoid regions. That value can then be used to generate the metric by multiplying with weight. At a high level, the total metric may be understood as indicated in Equation 1:

$$metric = \sum ( {v}_{target} * {w}_{T} - {v}_{avoid} * {w}_{A} - {v}_{sfm} * {w}_{B} ) \qquad Eq . 1$$

Where v_{target} is the volume of stimulated target tissue, v_{avoid} is the volume of the stimulated avoid region, and v_{sfm} is the total stimulated region at the optimized amplitude. In Equation 1, w_{A}, and w_{B} are as previously described, and each may be user adjustable; if desired, a target weight, w_{T}, (preset to 1 in some examples) may be included and would multiply with v_{target}. An analogous version of Equation 1 may also be used on a voxel by voxel basis to populate the Iₜₕ Metric Histogram 128.

Further examples and possible details for use in the preceding description of the algorithm in FIG. 4 may be found in U.S. Pat. No. 11,195,609, the disclosure of which is incorporated herein by reference for details of a voxelization, histogram, and optimization procedure. However, the preceding description is one way that optimization 120 can be implemented. In other examples, a different order of operations may be used. For example, in an alternative, with each given steering configuration, a seeking algorithm may be used to test different amplitude measures without generating a histogram at all, with the seeking algorithm used through several iterations until a current amplitude at a given steering configuration is determined that maximizes the metric as calculated above in Equation 1.

While a simplest approach to finding highest metrics may be to sweep all available steering and amplitude configurations (along with other parameters such as pulse width, shape, frequency, etc.), the computational burden of such a procedure may be excessive. It may be preferable to use a more selective approach. In some examples, for a given patient's anatomy, lead position, and medical condition, the optimization at 120 may use similarity analysis to identify similar patient characteristics in a database (such as “other data 108), and may use such analysis to generate starting points for the optimization 120. As noted, these are merely examples.

Those combinations of steering configurations and stimulation parameters that yield highest metrics may be characterized as candidate therapies, as indicated at 140. Candidate therapies 140 may further be rated or analyzed using secondary factors, such as power consumption. The candidate therapies may be presented to the physician. The physician can then select steering configurations and stimulation parameters for use in subsequent testing of the system. Testing can occur using an ETS or implantable pulse generator, as desired. If an ETS is used for testing, the pulse generator canister may be emulated by a cutaneous patch; otherwise, return current during ETS use goes through lead electrodes.

The preceding discussion presumes that all system electrodes are available. Thus, different steering configurations can be used to identify highest scoring metrics, however, it may be that one or more electrodes, as illustrated in FIG. 3, are not available. One approach is to modify block 122 as shown at 150, by limiting the electrode configurations that can used when determining a new steering or fractionalization at 122 in the iterations of the optimizer 120. That is, for example, the select steering configuration block 122 may use, for example and without limitation, a best-fit or cost-function type analysis to calculate a new fractionalization after electrode removal that recreates the electrical fields and/or therapy effects of the pre-candidate parameter set.

Another approach to the analysis with one or more electrodes unavailable is to add a block as shown at 160 to remove and re-calculate optimized parameters for one or more “pre-candidate” therapy parameter sets, which are then passed along as candidates to block 140. The removal and recalculation 160 may use, for example and without limitation, a best-fit or cost-function type analysis to calculate a new fractionalization after electrode removal that recreates the electrical fields and/or therapy effects of the pre-candidate parameter set.

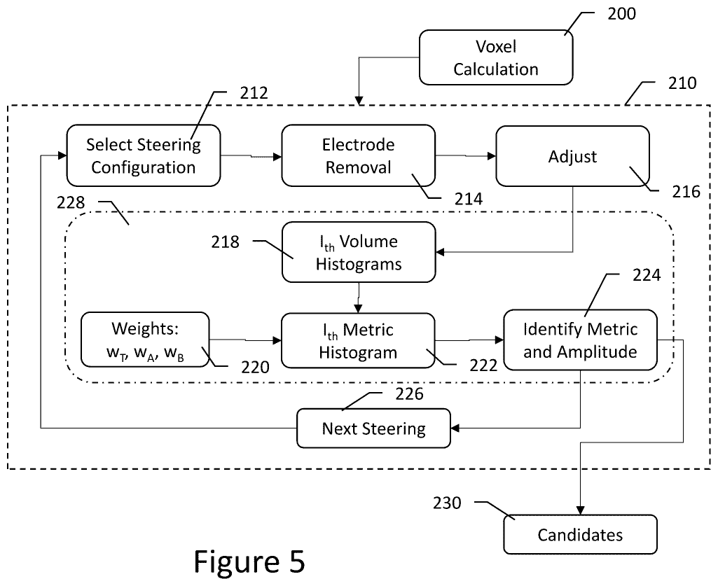

FIG. 5 illustrates another method in which optimization is performed with a newly reconfigured optimizer. Here, voxel calculation 200 occurs as was shown in FIG. 4, and the voxel calculation is passed to the optimizer 210. In the optimizer, steering configurations are selected as indicated at 212, similar to block 122 of FIG. 4 (omitting the alternative of block 150 in FIG. 4). After the steering configuration and fractionalization is generated at 212, a potential fractionalization is passed to block 214. At 214, any unavailable electrodes are removed from the fractionalization, generating a reduced fractionalization that is passed to block 216. The electrode removal block 214 may, for example, receive data from an implanted pulse generator or an ETS indicating electrodes that are unavailable due to high or low impedance (or any other reason), and/or from a clinician indicating electrodes that selected electrodes are not to be used and therefore unavailable.

Next, the fractionalization is adjusted, at indicated at 216. If no electrode removal is needed, the method may bypass blocks 214 and 216, as shown. Adjusting the fractionalization 216 can be performed by increasing the current on each available electrode in a proportional manner. A proportional approach may be referred to as normalization. This table shows a simplified example:

| Electrode | E1 | E2 | E3 | E4 | E5 |
|---|---|---|---|---|---|
| Original Fractionalization | 20% | 30% | 30% | 20% | 0 |
| Normalized Fractionalization | 29% | X | 43% | 29% | 0 |

Here, electrode E2 is not available as indicated by the X. The normalized fractionalization increases current in each of the active electrodes of the original fractionalization proportional to the original allocation of current, while the inactive electrodes (E5) remain set to zero. While a straight proportion-based approach is shown in the chart, other adjusting may occur, if desired. For example, a method may add current to electrodes based on proximity to the removed/unavailable electrodes using a Gaussian curve (or curves) centered on the removed/unavailable electrode(s) for the weighting functions. As a simple example, using a layout as in FIG. 3, the adjusted fractionalization may look different as shown in this chart:

| Electrode | E1 | E2 | E3 | E4 | E5 |
|---|---|---|---|---|---|
| Original Fractionalization | 20% | 30% | 30% | 20% | 0 |
| Adjusted Fractionalization | 30% | X | 47% | 23% | 0 |

Here, more of the redistributed current is allocated to E3, the one closest to E2, than to E1 and E4, which are farther away, and E1 receives more redistricted current than E4.

The adjusted fractionalization from block 216 is then passed to the Iₜₕ Volume Histogram calculation at 218, and the weights 220 are used to generate the Iₜₕ Metric Histogram at block 222, which is passed to block 224. Collectively, blocks 218, 220, 222, 224 may be described as a scoring block 228 for scoring the adjusted fractionalizations received from block 216. Blocks 218, 220, 222, 224, 226 and 230 may operate similar to blocks 124, 126, 128, 130, 132 and 140, respectively, of FIG. 4.

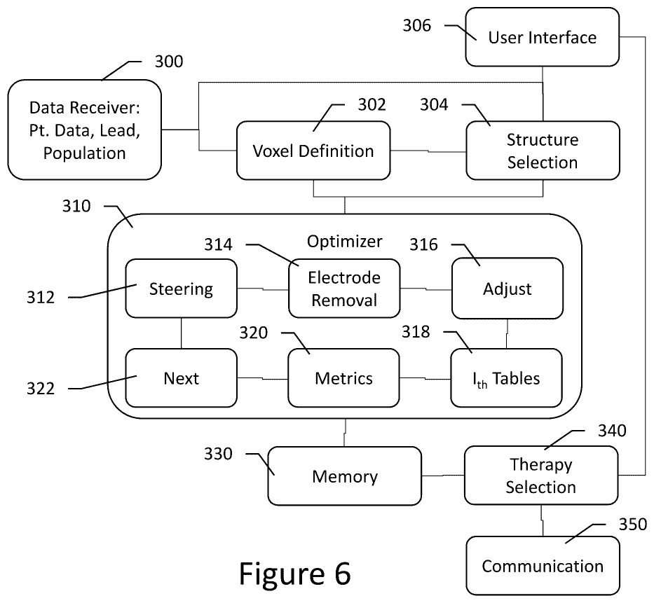

FIG. 6 shows an illustrative system. The system may include a data receiver block 300. For example, a communication circuit 350 may be coupled to a software or hardware module that is configured to receive and store information related to the patient anatomy, lead position, and/or other information including, for example, population-based structure data. Data input to 300 may also include inputs from a database of therapy settings of a previously programmed/treated population of patients, as desired. Also, brain functional data such as gathered using functional-based imaging, or electrophysiological activities recorded using an implanted lead, may also be used as data input at 300 as part of the overall mapping of “sweet” and “sour” spots-areas associated with therapeutic benefits and/or harms or side-effects. The data receiver 300 may, in addition or alternatively, receive SFM or other previously calculated therapy field or modelled data for the patient, which can be fed forward and used in the optimization procedure, as desired. Any previously created targets for the patient may be received in addition or as an alternative. Such data, in whole or in those parts available for a given patient (whether including or omitting anatomy data itself, or using previously generated target, SFM, or other modelled data) can be referred to as patient data received by the receiver module 300.

The data in block 300 is then used for voxel definition at 302, which may be implemented as another software module and/or by a separate or dedicated circuit (application specific integrated circuit, microcontroller, etc.). The voxel definition block 302 provides a voxelization to a structure selection block 304.

The structure selection block 304 is configured to receive user/physician inputs from a user interface 306, which may include one or more of a keyboard, mouse, trackball, touchscreen, monitor/output screen, voice or other audio input/output devices, etc. The structure selection block 304 may also receive structure data, such as would be helpful to identify structure boundaries as well as to identify structures, from the data receiver 300. The user interface 306 allows the user/physician to identify and select target and avoid regions in the patient anatomy for use by the structure selection block 304. If desired, block 304 may also limit which structures can be selected as targets, given a clinician's intended treatment and/or labeling limitations of the system.

The user interface is also used to allow the user/physician to select or modify the background, target, and/or and avoid structure weights that are used as illustrated above. In addition, the user interface may allow the physician to identify or select electrodes to use, or not to use, when performing the optimization process. Structure selection 304 may be applied to the voxel definitions to identify, on a structure-by-structure basis, the priority of stimulating (using for example, separate weights for each target structure) and/or avoiding (using for example, separate weights for each avoid structure) each structure. The effect of structure selection 304 is then to use the voxelization to determine the value of each voxel for each target and avoid structure. As used herein, the value of a voxel is as described above, to indicate each of the quantity of voxel fill by structures or portions of structures, such as probability shells, and probability of a neural response occurring if stimulated, and/or presence of a structure (whether target or avoid) in the voxel. The target and avoid structure data, along with voxel values corresponding thereto, are passed to the optimizer 310.

The optimizer 310 starts a series of iterative analyses with selecting a steering configuration at 312. Electrode removal occurs next, at block 314 (if any electrodes are unavailable) using, for example, indications from the physician provided via the user interface 306 and/or data received from the communication block at 350 indicating, from an implanted pulse generator or an ETS, electrodes that are unavailable. In some examples, the electrode removal module 314 is configured to identify an electrode that could be removed by identifying proximity to a neural structure less than a threshold proximity, and suggesting electrode removal to a physician via a user interface. In other examples, the electrode removal module is configured to identify an electrode that could be removed by identifying an electrode having an impedance outside of an impedance range, and suggesting electrode removal to a physician via a user interface.

The original fractionalization from block 312 is then adjusted as indicated at 316. The adjusting at 316 may include normalizing the data, or using a different adjustment such as one based on proximity to the removed/unavailable electrode.

The Iₜₕ tables are generated at 318, and metric/amplitude data is generated at 320. That is, the metric/amplitude data provides an indication, for a given steering configuration, of the pairings of metrics and amplitudes that would result. One or more best combinations are selected and stored to memory 330 by the optimizer 310. Absent exit conditions occurring, a next iteration is triggered at 322, and a new steering configuration is set at 312 and the process continues to iterate. Exit conditions and steering reconfiguration selections may be as described, for example, in U.S. Pat. No. 11,195,609, the disclosure of which is incorporated herein by reference.

When exit conditions are met, the set of data in memory 330 will provide one or more “best” or highest scoring steering settings and amplitude or parameter selections. These are then presented by a therapy selection module 340 to the user/physician via the user interface 306. The user/physician may then select or approve one or more proposed therapy. Therapy selection block 340 may generate an SFM for display via the user interface 306 to aid in the therapy selection process. Once a user has selected a therapy for implementation, a communications block 350 is used to transmit therapy parameters to a pulse generator or ETS. The pulse generator or ETS then delivers the selected therapy to the patient.

Steering selections at 312 may use an artificial intelligence method and/or a seeking function, with stop conditions predetermined. Block 310 may be configured to receive structure selection data that can inform the process of choosing steering configurations, in an example. For example, steering choices for the iterative process may use, without limitation, a database of steering selection configurations used for other, similar patients. By generically comparing the database of steering configurations to received structure selections, the optimizer 310 and steering selection block 312 may be able to eliminate a large portion of possible steering configurations quickly, to construct a shortened list. Some illustrative methods include iterative optimization methods such as gradient descent search, genetic algorithms, simulated annealing, random coordinate descent, particle swarm, fuzzy logic, among other machine learning search algorithms. Various details of the searching process are explained as well in U.S. Pat. No. 11,195,609, the disclosure of which is incorporated herein by reference.

While much of the above discussion focuses on use for DBS, other tissue regions can also be treated. For example, anatomical mapping may be used to identify neural and/or other structures to be targeted or avoided during other therapy, such as spinal cord stimulation (SCS), peripheral nerve stimulation, occipital nerve stimulation, muscles and muscle nerve fibers, therapies directed to the digestive tract, or other regions, and vagus nerve stimulation, As an example, in SCS, for example in the cervical spine, the spinal cord carries neural signals from various different parts of the body. As the science relating to spinal cord structure advances, knowledge may be obtained as to which parts of the spinal cord at a given vertebral level carry signals to and from what part of the body. With this knowledge, therapy and avoid regions in the spinal cord may be defined/determined. If a plurality of leads are present, or a paddle lead, at the given vertebral level, a steering and SFM model may be determined for the given vertebral level using a spinal cord atlas as well as lead implantation/position data (such as from an X-ray or other imaging system). A similar process as just described may be used to define therapy that targets the portion of the spinal cord carrying neural signals (such as pain signaling) that are to be interfered with, while other portions (such as carrying motor signals) are to be avoided. The metric calculations described above can then be used to optimizer steering to limit side effects and achieve desired therapy.

Turning back to FIG. 6, data receiver 300 and/or communication block 350 may include a communications circuit (a transceiver, antenna or the like, for example, Bluetooth, WiFi, Medradio, etc.) and/or inputs/output circuits for use with, for example, local area network cables. In some examples, on the other hand, the data receiver 300 is instead a software module that communicates with other applications in the CP; communication via external hardware can be optional for data receiver 300. A microcontroller or microprocessor with particularly configured software or other instructions stored therewith, for example on non-transient memory, such as memory 330 which may include Flash, RAM, ROM, etc., as are known in the art. Overall the implementation in FIG. 6 may be on a tablet or laptop computer, such as a clinician programmer or CP as described above.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A configuration system for configuring delivery of neuromodulation to specific tissue of a patient by an implantable system having a plurality of electrodes, the configuration system comprising:
a receiver module configured to receive at least patient data for a patient and lead position indicating a location of a lead carrying at least two of the plurality of electrodes thereon relative to neural tissue of the patient;
an optimizer for identifying candidate therapy parameter sets indicating utilization of the plurality of electrodes during therapy, the optimizer comprising:
a steering module for identifying a potential fractionalization to analyze, the potential fractionalization setting proportions of total current to be issued by each of the electrodes;
an electrode removal module that receives the potential fractionalization and removes unavailable electrodes from the potential fractionalization to generate a reduced fractionalization;
an adjusting module that receives the reduced fractionalization and creates an adjusted fractionalization;
a scoring module that receives the adjusted fractionalization and calculates a plurality of metrics for the adjusted fractionalization using a plurality of amplitude settings; and
a candidate module that identifies one or more candidate therapies using the plurality of metrics.

2. The configuration system of claim 1, wherein the optimizer is configured to use the steering module to identify a plurality of potential fractionalizations.

3. The configuration system of claim 1, wherein the potential fractionalization identifies active and inactive electrodes, and the adjusting module creates the adjusted fractionalization by proportionally increasing a fraction of current to be delivered by each active electrode which is not removed by the electrode removal module.

4. The configuration system of claim 1, wherein the potential fractionalization identifies active and inactive electrodes, and the adjusting module modifies current fractionalization by adding current to active electrodes in proportion to proximity to the unavailable electrodes.

5. The configuration system of claim 4, wherein the proportion is based on a Gaussian curve.

6. The configuration system of claim 1, wherein the electrode removal module removes electrodes having an impedance outside of an impedance range.

7. The configuration system of claim 1, wherein the electrode removal module removes electrodes that have been identified as unavailable by a physician.

8. The configuration system of claim 7, wherein the electrode removal module is configured to identify an electrode that could be removed by identifying proximity to a neural structure less than a threshold proximity, and suggesting electrode removal to a physician via a user interface.

9. The configuration system of claim 7, wherein the electrode removal module is configured to identify an electrode that could be removed by identifying an electrode having an impedance outside of an impedance range, and suggesting electrode removal to a physician via a user interface.

10. The configuration system of claim 1, further comprising a voxel calculation module which identifies volumes located around the lead as voxels, and labels at least some voxels as target voxels to which therapy is to be directed.

11. The configuration system of claim 10, wherein the voxel calculation module also labels at least some voxels as avoid voxels to which therapy is to be avoided.

12. The configuration system of claim 10, wherein the scoring module calculates the plurality of metrics by determining which of the voxels would be activated by the adjusted fractionalization at a plurality of current amplitudes.

13. The configuration system of claim 10, wherein the scoring module uses one or more of a weight for target voxels, a weight for avoid voxels, a weight for total activated voxels and/or a weight for total activated non-target and non-avoid voxels to calculate the metrics.

14. A method of operation in a neuromodulation system, the neuromodulation system including an implantable system having a plurality of electrodes, and a configuration system configured to perform steps of the method, the method comprising:
a) receiving at least patient data for a patient and a lead position indicating a location of a lead carrying at least two of the plurality of electrodes thereon relative to neural tissue of the patient;
b) identifying a potential fractionalization to analyze, the potential fractionalization setting proportions of total current to be issued by each of the electrodes;
c) removing unavailable electrodes from the potential fractionalization to generate a reduced fractionalization;
d) adjusting the reduced fractionalization to create an adjusted fractionalization;
e) calculating a plurality of metrics for the adjusted fractionalization using a plurality of amplitude settings; and
identifying one or more of the adjusted fractionalizations as one or more candidate therapies using the plurality of metrics.

15. The method of claim 14, further comprising communicating the one or more candidate therapies from the configuration system to a pulse generator for the implantable system, and activating the pulse generator to issue neuromodulation to the patient using at least one of the one or more candidate therapies.

16. The method of claim 14, further comprising identifying a plurality of potential fractionalizations, and performing each of c), d) and e) for each of the plurality of potential fractionalizations.

17. The method of claim 14, wherein the potential fractionalization identifies active and inactive electrodes, and step d) includes proportionally increasing a fraction of current to be delivered by each active electrode which is has not removed during step c).

18. The method of claim 14, wherein the potential fractionalization identifies active and inactive electrodes, and step d) include increasing proportions of total current to be issued by each of the electrodes in proportion to proximity to the unavailable electrodes.

19. The method of claim 14, wherein step c) includes removing electrodes having an impedance outside of an impedance range.

20. The method of claim 14, wherein step c) includes removing electrodes that have been identified as unavailable by a physician.
